(19) United States
(12) Patent Application Publication
(10) Pub. No.: US 2020/0269280 A1
(43) Pub. Date: Aug. 27, 2020

(54) ULTRASOUND DEVICE WITH PIEZOELECTRIC MICROMACHINED ULTRASONIC TRANSDUCERS

(71) Applicant: Butterfly Network, Inc., Guilford, CT (US)

(72) Inventors: Jonathan M. Rothberg, Guilford, CT (US); Keith G. Fife, Palo Alto, CA (US); Gerard Schmid, Guilford, CT (US)

(73) Assignee: Butterfly Network, Inc., Guilford, CT (US)

(21) Appl. No.: 15/930,403

(22) Filed: May 12, 2020

Related U.S. Application Data
(63) Continuation of application No. PCT/US2018/061296, filed on Nov. 15, 2018.
(60) Provisional application No. 62/586,795, filed on Nov. 15, 2017.

(51) Int. Cl. B06B 1/06; B06B 1/02; H01L 41/253; H01L 41/332; H01L 41/313; A61B 8/00

(52) U.S. Cl. CPC B06B 1/064; B06B 1/0215; H01L 41/253; G01N 29/2437; H01L 41/313; A61B 8/4494; H01L 41/332

(57) ABSTRACT

Ultrasound devices including piezoelectric micromachined ultrasonic transducers (PMUTs) are described. Frequency tunable PMUT arrays are provided. The PMUTs may be formed on the same substrate or a different substrate than an integrated circuit substrate. The PMUTs may be formed in a variety of ways and from various suitable piezoelectric materials.

### CROSS-REFERENCE TO RELATED APPLICATIONS

This application is a Continuation of International Patent Application Serial No. PCT/US2018/061296, filed Nov. 15, 2018, under Attorney Docket No. B1348.70067WO00 and entitled “ULTRASOUND DEVICE WITH PIEZOELECTRIC MICROMACHINED ULTRASONIC TRANSDUCERS,” which is hereby incorporated herein by reference in its entirety.

Patent Application Serial No. PCT/US2018/061296 claims the benefit under 35 U.S.C. § 119(e) of U.S. Provisional Patent Application Ser. No. 62/586,795, filed Nov. 15, 2017 under Attorney Docket No. B1348.70067US00 and entitled “ULTRASOUND DEVICE WITH PIEZOELECTRIC MICROMACHINED ULTRASONIC TRANSDUCERS,” which is hereby incorporated herein by reference in its entirety.

### BACKGROUND

### Field

The present application relates to ultrasound devices including piezoelectric ultrasonic transducers.

### Related Art

Ultrasound devices conventionally include macro-scale piezoelectric crystal transducers. The crystal transducers are formed and individually placed on a board to create an array.

### BRIEF SUMMARY

According to an aspect of the present application, an ultrasound device is provided, comprising an integrated multi-substrate die including a piezoelectric micromachined ultrasonic transducer (PMUT) substrate, a transmit circuitry substrate, and a receive circuitry substrate coupled together.

According to an aspect of the present application, an ultrasound device is provided, comprising an integrated multi-substrate die including a piezoelectric micromachined ultrasonic transducer (PMUT) substrate coupled with an integrated circuit (IC) substrate.

According to an aspect of the present application, an ultrasound device is provided, comprising a substrate, an integrated circuit formed in the substrate, and a layer of thin film piezoelectric micromachined ultrasonic transducers (PMUTs) integrated with the substrate.

### BRIEF DESCRIPTION OF DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 illustrates an ultrasound device comprising a layer of piezoelectric micromachined ultrasonic transducers (PMUTs) formed on and integrated with an integrated circuit substrate comprising integrated circuitry.

FIG. 2 is a flowchart illustrating a method of fabricating the ultrasound device of FIG. 1.

FIG. 3 is a flowchart illustrating an example of the stage of forming a piezoelectric thin film from the method of FIG. 2.

FIGS. 4A-4E illustrate a fabrication sequence for forming thin film PMUTs, according to a non-limiting embodiment of the present application.

FIG. 5 illustrates an ultrasound device comprising a PMUT substrate bonded with an integrated circuit substrate, according to a non-limiting embodiment.

FIG. 6 is a flowchart illustrating a method of fabricating the ultrasound device of FIG. 5.

FIG. 7 illustrates an ultrasound device comprising a PMUT substrate, a transmit circuitry substrate, and a receive circuitry substrate, according to a non-limiting embodiment.

FIG. 8 is a flowchart illustrating a method of fabricating the ultrasound device of FIG. 7.

FIG. 9 illustrates a system architecture of an ultrasound device comprising an array of PMUTs, according to a non-limiting embodiment.

FIG. 10 illustrates an array of PMUTs, according to a non-limiting embodiment.

FIG. 11A is a top view of a PMUT having multiple electrodes configured to provide selectable frequency operation of the PMUT, according to a non-limiting embodiment.

FIG. 11B is a cross-sectional view of the PMUT of FIG. 11A.

FIGS. 11C-11H illustrate a fabrication sequence for fabricating PMUTs according to a non-limiting embodiment.

FIGS. 11I-11J illustrate PMUT arrays according to a non-limiting embodiment.

FIG. 12 illustrates an array of PMUTs of different types having different dimensions and frequencies of operation, according to a non-limiting embodiment.

FIG. 13 is a cross-sectional view of a group of PMUTs having different thicknesses and frequencies of operation, according to a non-limiting embodiment.

FIGS. 14A-14B illustrate a handheld device comprising an ultrasound probe and a display, in accordance with some embodiments of the technology described herein.

FIG. 15 is a diagram illustrating a handheld probe comprising an ultrasound probe, in accordance with some embodiments of the technology described herein.

FIGS. 16A-16B illustrate a patch comprising an ultrasound probe, in accordance with some embodiments of the technology described herein.

FIG. 17 illustrates a pill comprising an ultrasound probe, in accordance with some embodiments of the technology described herein.

### DETAILED DESCRIPTION

Aspects of the present application provide ultrasound devices comprising thin film piezoelectric micromachined ultrasonic transducers (PMUTs). The PMUTs may be coupled to integrated circuitry configured to control their operation. In some embodiments, the PMUTs are formed integrally on the same substrate as the integrated circuitry. In other embodiments, a PMUT substrate includes the PMUTs and an integrated circuit (IC) substrate includes the integrated circuitry, and the two are bonded together. According to a further embodiment, a PMUT substrate includes the PMUTs, a first integrated circuit substrate includes transmit circuitry, and a second integrated circuit substrate includes receive circuitry, and the three substrates are coupled together.

The PMUTs may be formed into an array or other suitable arrangement, and the array may be frequency tunable. In one embodiment, the plurality of PMUTs include a single type of PMUT which is frequency controllable through excitation of a selected subset of excitation electrodes. According to another embodiment, the array of PMUTs includes different types of PMUTs having different operational frequencies. The different operational frequencies may be provided by the different dimensions of the different types of PMUTs.

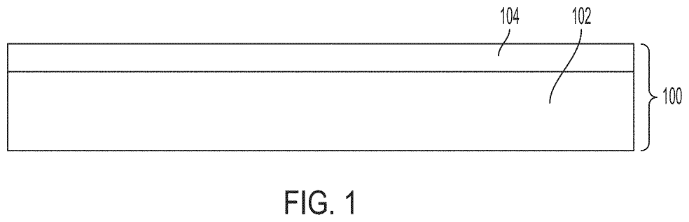

According to aspects of the present application, an ultrasound device having PMUTs may be formed using different configurations and techniques, including the number and arrangement of substrates and/or wafers used. FIG. 1 shows ultrasound device 100 having PMUTs formed integrally on the same substrate as the integrated circuitry. Ultrasound device 100 includes PMUT layer 104 formed on and integrated with integrated circuit substrate 102. PMUT layer 104 may include any suitable piezoelectric material, including aluminum nitride or vapor deposited lead zirconate titanate (PZT). PMUT layer 104 may be a thin film having any suitable dimensions and any suitable number of PMUTs arranged on substrate 102. Substrate 102 may include integrated circuitry configured to electrically couple with the PMUTs in PMUT layer 104. Substrate 102 may be any suitable semiconductor die (e.g., silicon die, or a complementary metal-semiconductor-oxide (CMOS) die). Although only substrate 102 and PMUT layer are shown, it should be appreciated that other layers may be formed between PMUT layer 104 and substrate and/or over PMUT layer 104. For example, some embodiments may include an underlayer, such as a seed layer and/or buffer layer, formed between substrate 102 and PMUT layer 104. Electrodes may be considered part of the PMUT layer, or alternatively may be considered separate and may be formed above and below the PMUT layer.

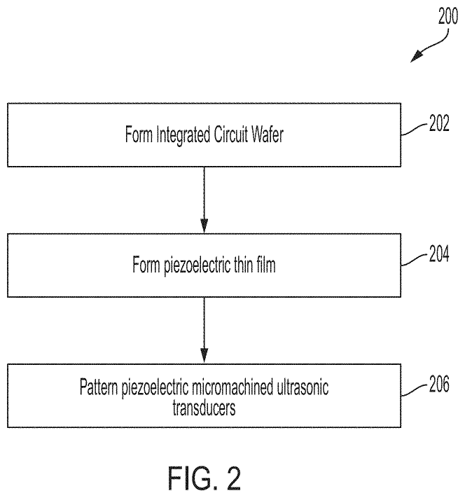

FIG. 2 illustrates a method fabricating an ultrasound device having PMUTs integrally formed on a substrate, such as the ultrasound device of FIG. 1. Method 200 includes step 202 of forming an integrated circuit wafer, which may include forming circuitry coupled to the PMUTs in the resulting ultrasound device. Method 200 proceeds to step 204, which includes forming a piezoelectric thin film on the wafer. Examples of piezoelectric materials that may form the piezoelectric thin film include aluminum nitride and vapor deposited lead zirconate titanate (PZT). Formation of the piezoelectric film may include fabrication of the film using suitable parameters (e.g., temperature, deposition rate) that allow the piezoelectric film to be formed having a desired degree and type of crystallinity as well as piezoelectric properties. In some embodiments, temperature and deposition rate may be selected to allow for rearrangement and/or diffusion of the piezoelectric material to improve crystallinity and reduce phase separation of the resulting film. Examples of deposition techniques that may be used to form the piezoelectric thin film include a physical vapor deposition (PVD) process, a sputtering process, an atomic layer deposition (ALD) process, and a chemical vapor deposition (CVD) process. In some embodiments, deposition techniques that can be performed at lower temperatures may be used to form the piezoelectric thin film, including pulsed laser deposition, metal-organic chemical vapor deposition (MOCVD), plasma-enhanced chemical vapor deposition (PECVD), and UV-enhanced deposition. In some embodiments, the piezoelectric thin film may be formed by spin-coating from a sol-gel solution followed by curing processes. The piezoelectric film may be formed in one or more steps to build up the desired thickness of the layer.

To reduce or prevent damage to the circuitry in the underlying wafer, step 204 may include forming the piezoelectric thin film below temperatures where such damage may occur. For example, some embodiments may include fabrication techniques that involve forming the film at temperatures below 450° C. Such techniques may be particularly suitable when the integrated circuit wafer is a CMOS wafer, since damage to CMOS wafers is more likely to occur when the wafer is at or exceeds 450° C.

In some embodiments, forming the piezoelectric thin film may include forming an underlayer (e.g., buffer layer, seed layer) on the integrated circuit wafer and forming the piezoelectric film over the underlayer. In some embodiments, the underlayer may crystallize on the wafer, such as at suitable semiconductor-compatible processing temperatures, and promote a desired degree and/or type of crystallinity of the piezoelectric film. In some embodiments, the underlayer may reduce or prevent diffusion of material of the piezoelectric film into the underlying wafer. Examples of suitable materials that may be used to form an underlayer may include TiO₂, PbO, and PbTiO₃.

Method 200 then proceeds to step 206, which includes patterning the piezoelectric thin film to form the PMUTs. Any suitable lithography techniques may be used in patterning the piezoelectric thin film to form the PMUTs. It should be appreciated that method 200 may be performed on a wafer, which may be subsequently diced to form individual die. A resulting die may include PMUTs and circuitry coupled to the PMUTs, and may be used in an ultrasound device.

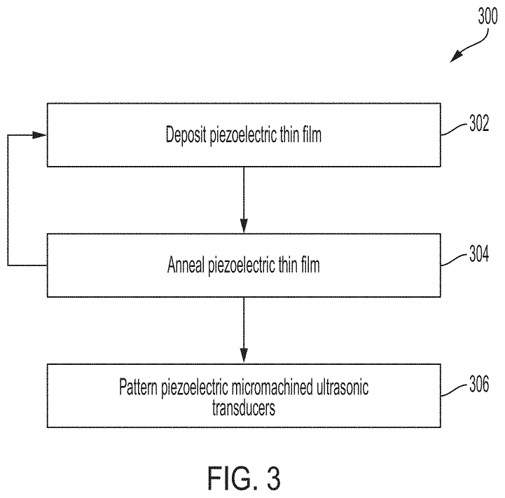

Some embodiments for forming PMUTs may involve using an anneal process as part of forming the piezoelectric thin film. FIG. 3 illustrates a method of forming a piezoelectric thin film and patterning the film to form PMUTs, which may be implemented as part of steps 204 and 206 of method 200 shown in FIG. 2. Method 300 includes depositing piezoelectric thin film by step 302, which may include depositing the piezoelectric thin film over a substrate having integrated circuitry. Method 300 next proceeds by step 304, which includes applying an annealing process to the deposited piezoelectric thin film. The annealing process may involve using a technique that selectively increases the temperature of the piezoelectric thin film while minimizing any increase in temperature at the wafer. In some embodiments, a laser anneal process may be implemented as part of step 304. For example, a laser pulse may be applied at or near the surface of the deposited film, which will raise the temperature of the film for a duration of time while the wafer, including the circuitry formed in the wafer, may experience only a small increase in temperature in comparison to the film. The laser used for performing this selective anneal process may emit light that is highly absorbed by the piezoelectric material(s) of the film, and in embodiments that include an underlayer, by the underlayer. Although FIG. 3 shows steps 302 and 304 as separate steps, it should be appreciated that an anneal process may be performed during deposition of the piezoelectric film. For example, a laser pulse may be applied during deposition of the film to selectively increase the temperature where the film is being deposited.

As shown in FIG. 3, the deposition step of 302 and the anneal step of 304 may be iterative, and may be repeated any suitable number of times to form a piezoelectric film having a desired thickness. After the film has been formed, method 300 proceeds to step 306, which includes patterning the film to form the PMUTs. The wafer may be subsequently diced to from individual die having the PMUTs.

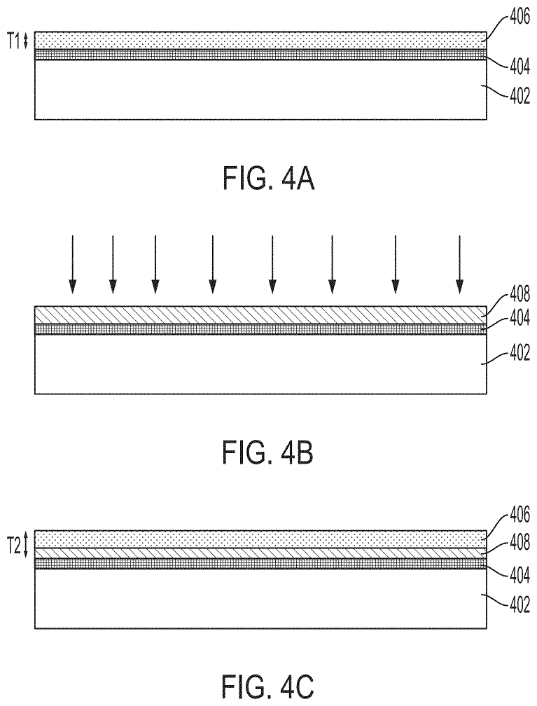

FIGS. 4A-4E illustrate a fabrication sequence for forming thin film PMUTs. Although the fabrication process is shown at the die level, it should be appreciated that the fabrication may be applied for an entire wafer. FIG. 4A illustrates substrate 402, bottom electrode 404 formed over substrate 402, and piezoelectric film 406 formed over bottom electrode 404. Piezoelectric film 406 may be formed using any suitable techniques discussed herein, including techniques described in connection with FIGS. 2 and 3. The piezoelectric film 406 may have a thickness T1. FIG. 4B shows the application of an anneal process, such as an anneal process used in step 304 of method 300, to piezoelectric film, which may alter the characteristics (e.g., crystallinity degree and/or type) of the piezoelectric film, to form annealed piezoelectric film 408 of thickness T1. FIG. 4C shows additional piezoelectric film 406 formed over the annealed film 408. Thus, the total piezoelectric thin film may now have thickness T2. FIG. 4D shows an additional anneal process, such as an anneal process used in step 304 of method 300, to form an annealed piezoelectric film 408 of thickness T2. As discussed above in connection with FIG. 3, multiple deposition and anneal steps may be repeated any suitable number of times to form a piezoelectric film having a desired thickness.

As shown in FIG. 4E, the piezoelectric film may be patterned into regions 408a, 408b, and 408c forming the resulting PMUTs and top electrodes 410a, 410b, 410c may be formed over regions 408a, 408b, 408c. In some embodiments, forming the PMUTs may include forming a top electrode layer over the piezoelectric film and patterning the film and the top electrode layer to form the individual PMUTs. It can be seen that the type of PMUTs illustrated lack a transducing gap. The excitation electrodes are positioned in contact with the top and bottom surfaces of the piezoelectric layer in the illustrated example. In some embodiments, a layer of thin film PMUTs may be provided without a transducing gap. In other embodiments, a layer of thin film PMUTs may be provided with a transducing gap.

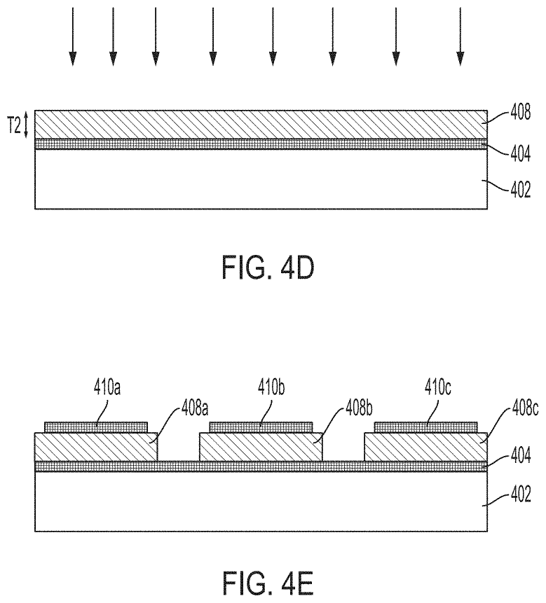

In some embodiments, an ultrasound device may have a multi-stacked die configuration, including a substrate having PMUTs bonded to a substrate having integrated circuitry. FIG. 5 shows an ultrasound device comprising a PMUT substrate 504 bonded with an integrated circuit substrate 502 through bonding points 506. In some embodiments, the PMUTs of PMUT substrate 504 may be arranged on a substrate (e.g., semiconductor die) such that the PMUTs are on a surface of the substrate distal from the integrated circuit substrate 502. In other embodiments, the PMUTs of PMUT substrate 504 may be arranged on a surface of a substrate proximate to the integrated circuit substrate 502. Any suitable technique for bonding two substrates may be used to bond PMUT substrate 504 with an integrated circuit substrate including bonding pillars, contact pads, and wire bonds. Bonding points 506 may electrically couple individual PMUTs (not shown) on substrate 504 to control circuitry on integrated circuit substrate 502.

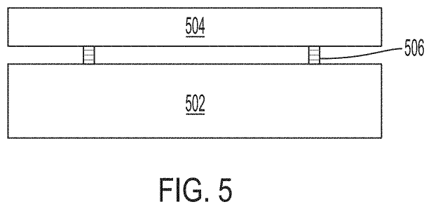

FIG. 6 is a flowchart illustrating a method 600 of fabricating the ultrasound device of FIG. 5. Step 602 of method 600 includes forming a PMUT wafer, which may include forming PMUTs arranged on a surface of a substrate. Step 602 may include forming and patterning piezoelectric and/or electrode layers to form individual PMUTs. Step 604 of method 600 includes forming an integrated circuit wafer. Step 606 includes bonding the PMUT wafer with the integrated circuit wafer. Steps 602 and 604 may be performed sequentially (performing step 602 followed by 604, or vice versa), simultaneously, or in any suitable order. In some embodiments, steps 602 and 604 may be performed in parallel. In some embodiments, steps 602 and 604 may be performed in separate facilities (e.g., separate fabrication facilities) and/or by separate entities. Performing steps 602 and 604 in parallel may be beneficial in some embodiments as providing separate wafer supplies.

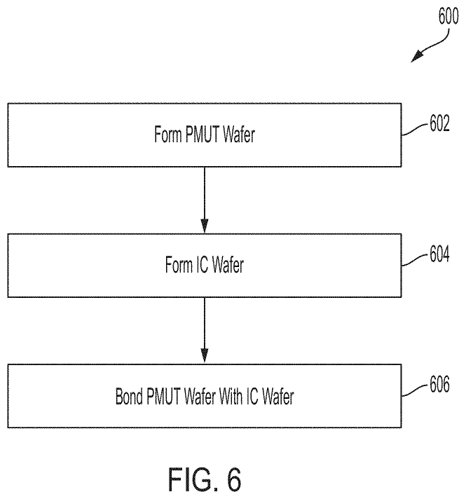

In some embodiments, an ultrasound device may include multiple wafers including a PMUT wafer, a transmit circuitry wafer, and a receive circuitry wafer. FIG. 7 shows a block diagram of an ultrasound device 700 including three substrates bonded together. The ultrasound device includes a first substrate 702, a second substrate 704, and a third substrate 706. The first substrate 702, the second substrate 704, and the third substrate 706 may be, for example, wafers or dies, and each substrate may include multiple layers of materials (e.g., silicon, oxides, metals, etc.). The bottom surface of the first substrate 702 is bonded to the top surface of the second substrate 704. The bottom surface of the second substrate 704 is bonded to the top surface of the third substrate 706. The bonding between the first substrate 702 and the second substrate 704 and the bonding between the second substrate 704 and the third substrate 706 may include, for example, thermal compression (also referred to herein as “thermocompression”), eutectic bonding, silicide bonding (which is a bond formed by bringing silicon of one substrate into contact with metal on a second substrate under sufficient pressure and temperature to form a metal silicide, creating a mechanical and electrical bond), or solder bonding.

The ultrasound device 700 is configured to drive ultrasound transducers to emit pulsed ultrasonic signals into a structure, such as a patient. The pulsed ultrasonic signals may be back-scattered from structures in the body, such as blood cells or muscular tissue, to produce echoes that return to the ultrasound transducers. These echoes may then be converted into electrical signals by the transducer elements. The electrical signals representing the received echoes are then converted into ultrasound data.

The first substrate 702 includes the ultrasonic transducers, in the form of thin film PMUTs. The second substrate 704 includes integrated transmit circuitry, which may include one or more pulsers configured to receive waveforms from one or more waveform generators and output driving signals corresponding to the waveforms to the ultrasonic transducers. The third substrate 706 includes integrated receive circuitry, which may be integrated analog receive circuitry and/or integrated digital receive circuitry, and which may be configured to receive and process electronic signals generated by the ultrasonic transducers when impinged upon by acoustic signals. For example, the analog receive circuitry may include amplifiers configured to amplify the analog electronic signals generated by the ultrasonic transducers and/or analog-to-digital converters configured to convert the amplified analog signals to digital signals. The digital processing circuitry may include, for example, image formation circuitry configured to generate ultrasound images from the digitally converted electronic signals generated by the ultrasonic transducers.

The second substrate 704 may be implemented in a different microfabrication technology node than the third substrate 706, and the technology node of the third substrate 706 may be a more advanced technology node with smaller feature sizes than the technology node in which the second substrate 704 is implemented. For example, the technology node of the second substrate 704 may be a technology node that provides circuit devices (e.g., transistors) capable of operating at voltages in the range of approximately 80-200 V, such as 80 V, 90 V, 100 V, 200 V, or >200 V. In some embodiments, the technology node of the second substrate 704 may be a technology node that provides circuit devices (e.g., transistors) capable of operating at other voltages, such as voltages in the range of approximately 5-30 V or voltages in the range of approximately 30-80V. By operating at such voltages, circuitry in the second substrate 704 may be able to drive the ultrasound transducers in the first substrate 702 to emit acoustic waves having acceptably high pressures. The technology node of the second substrate 704 may be, for example, 65 nm, 80 nm, 90 nm, 110 nm, 130 nm, 150 nm, 180 nm, 220 nm, 240 nm, 250 nm, 280 nm, 350 nm, 500 nm, >500 nm, or any other suitable technology node.

The technology node of the third substrate 706, for example, may be one that provides circuit devices (e.g., transistors) capable of operation at a voltage in the range of approximately 0.45-0.9V, such as 0.9V, 0.85V, 0.8V, 0.75V, 0.7V, 0.65V, 0.6V, 0.6V, 0.55V, 0.5V, and 0.45V. In some embodiments, the technology node of the third substrate 706 may be one that provides circuit devices capable of operation at a voltage in the range of approximately 1-1.8 V, or approximately 2.5-3.3 V. By operating at such voltages, power consumption of circuitry in the third substrate 706 may be reduced to an acceptable level. Additionally, the feature size of devices provided by the technology node may enable an acceptably high degree of integration density of circuitry in the third substrate 706. The technology node of the third substrate 706 may be, for example, 90 nm, 80 nm, 65 nm, 55 nm, 45 nm, 40 nm, 32 nm, 28 nm, 22 nm, 20 nm, 16 nm, 14 nm, 10 nm, 7 nm, 5 nm, 3 nm, etc.

In some embodiments, the second substrate 704 includes power management circuitry, such as low-dropout regulators, multi-level pulsers, and/or charge recycling circuitry. For further discussion of multi-level pulsers and charge recycling circuitry, see U.S. Pat. No. 9,492,144 titled “MULTI-LEVEL PULSER AND RELATED APPARATUS AND METHODS,” granted on Nov. 15, 2016, and U.S. patent application Ser. No. 15/087,914 titled “MULTILEVEL BIPOLAR PULSER,” issued as U.S. Pat. No. 10,082,565, each of which is assigned to the assignee of the instant application and each of which is incorporated by reference herein in its entirety. Including such circuitry in the second substrate 704 rather than an external printed circuit board may reduce the size of the final ultrasound system including the ultrasound device 700.

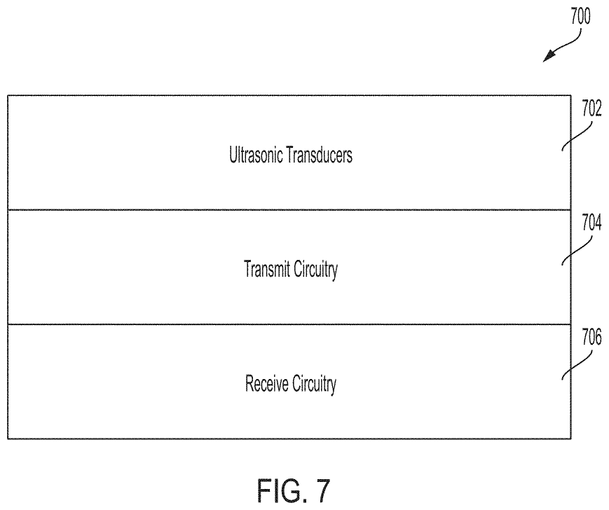

FIG. 8 is a flowchart illustrating a method of fabricating the ultrasound device of FIG. 7. Step 802 of method 800 includes forming a PMUT wafer, which may include forming PMUTs arranged on a surface of a substrate. Step 804 of method 800 includes forming a transmit integrated circuit wafer. Step 806 of method 800 includes forming a receive integrated circuit wafer. Step 808 of method 800 includes bonding the PMUT wafer with either the transmit integrated circuit wafer or the receive integrated circuit wafer. Step 810 of method 800 includes bonding the remaining transmit or receive wafer with the two bonded wafers.

Steps 802, 804, and 806 may be performed sequentially (with those three steps arranged in any order), simultaneously, or in any suitable order. In some embodiments, steps 802, 804, and 806 may be performed in parallel. In some embodiments, two or more of steps 802, 804, and 806 may be performed in separate facilities (e.g., separate fabrication facilities) and/or by separate entities. Performing steps 802, 804, and 806 in parallel may be beneficial in some embodiments as providing separate wafer supplies.

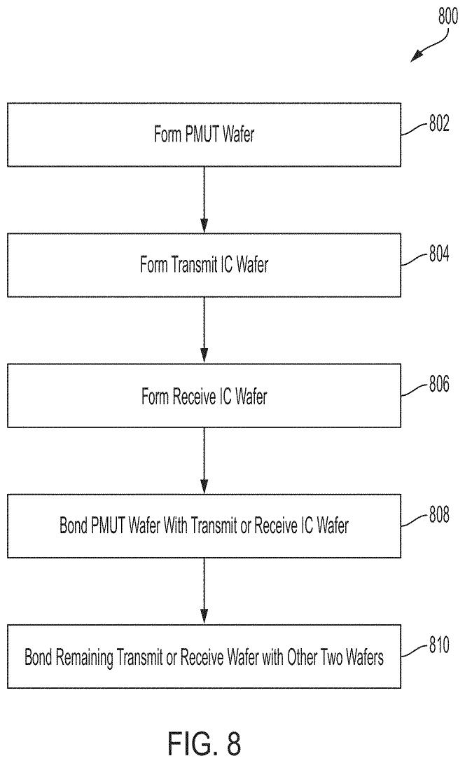

FIG. 9 shows an illustrative example of a monolithic ultrasound device 900 embodying various aspects of the technology described herein. The ultrasound device 900 may be a solid state device in some embodiments. The ultrasound device may include or define a chipset in some embodiments. As shown, the device 900 may include one or more transducer arrangements (e.g., arrays) 902, transmit (TX) circuitry 904, receive (RX) circuitry 906, a timing & control circuit 908, a signal conditioning/processing circuit 910, a power management circuit 918, and/or a high-intensity focused ultrasound (HIFU) controller 920. In the embodiment shown, all of the illustrated elements are formed on a single semiconductor die 912. It should be appreciated, however, that in alternative embodiments one or more of the illustrated elements may be instead located off-chip, for example in the 2-die and 3-die configurations described above. In addition, although the illustrated example shows both TX circuitry 904 and RX circuitry 906, in alternative embodiments only TX circuitry or only RX circuitry may be employed. For example, such embodiments may be employed in a circumstance where one or more transmission-only devices 900 are used to transmit acoustic signals and one or more reception-only devices 900 are used to receive acoustic signals that have been transmitted through or reflected off of a subject being ultrasonically imaged.

It should be appreciated that communication between one or more of the illustrated components may be performed in any of numerous ways. In some embodiments, for example, one or more high-speed busses (not shown), such as that employed by a unified Northbridge, or one or more high-speed serial links (e.g. 1 Gbps, 2.5 Gbps, 5 Gbps, 10 Gbps, 20 Gbps) with any suitable combined bandwidth (e.g. 10 Gbps, 20 Gbps, 40 Gbps, 60 Gbps, 80 Gbps, 100 Gbps, 120 Gbps, 150 Gbps, 240 Gbps) may be used to allow high-speed intra-chip communication or communication with one or more off-chip components. In some embodiments, communication with off-chip components may be performed and may be in the analog domain, using analog signals.

The one or more transducer arrays 902 may take on any of numerous forms, and aspects of the present technology do not necessarily require the use of any particular type or arrangement of transducer cells or transducer elements. Indeed, although the term “array” is used in this description, it should be appreciated that in some embodiments the transducer elements may not be organized in an array and may instead be arranged in some non-array fashion. In various embodiments, each of the transducer elements in the array 902 may, for example, include one or more thin film PMUTs. In some embodiments, the transducer elements of the transducer array 902 may be formed on the same chip as the electronics of the TX circuitry 904 and/or RX circuitry 906 or, alternatively integrated onto the chip having the TX circuitry 904 and/or RX circuitry 906. In still other embodiments, the transducer elements of the transducer array 902, the TX circuitry 904 and/or RX circuitry 906 may be tiled on multiple chips.

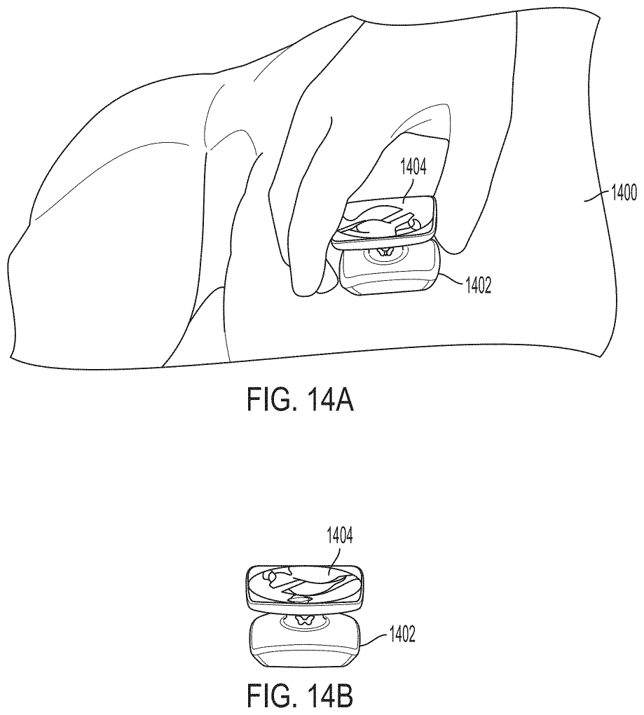

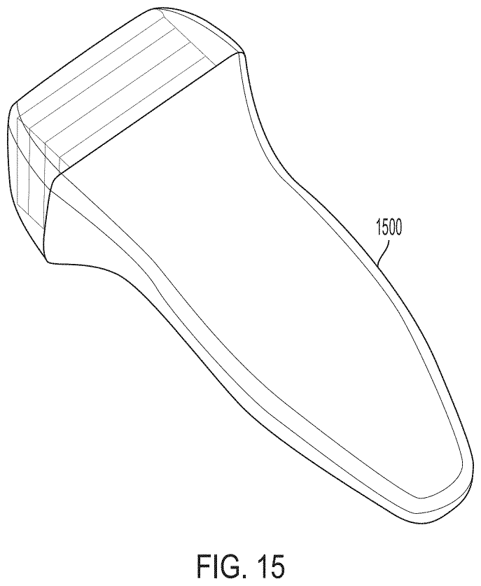

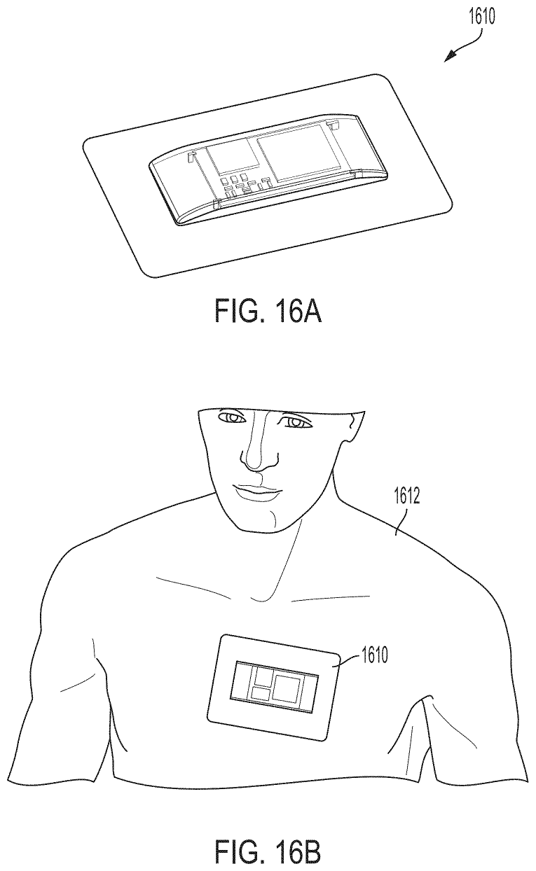

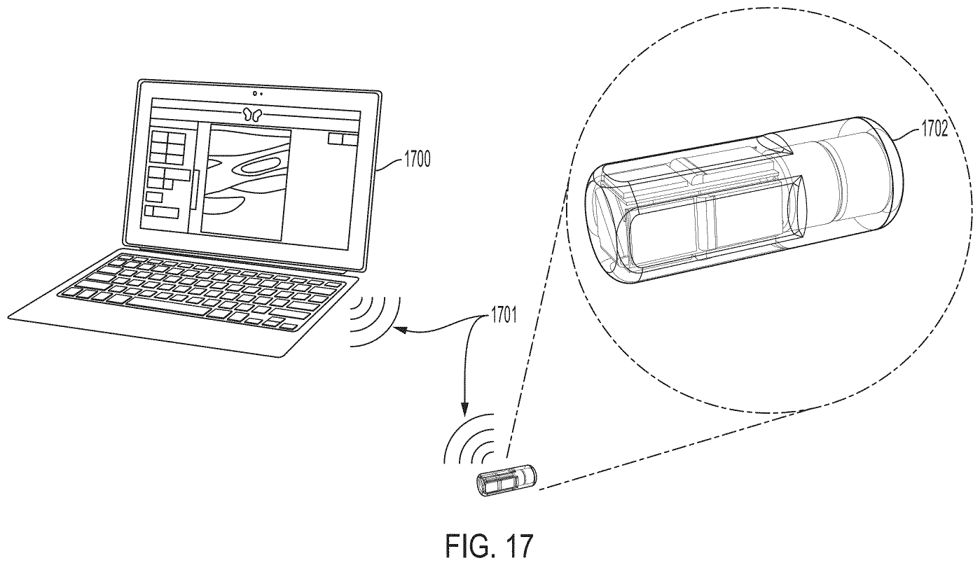

The transducer array 902, TX circuitry 904, and RX circuitry 906 may be, in some embodiments, integrated in a single ultrasound probe. In some embodiments, the single ultrasound probe may be a hand-held probe including, but not limited to, the hand-held probes described below with reference to FIGS. 14A-14B and 15. In other embodiments, the single ultrasound probe may be embodied in a patch that may be coupled to a patient. FIGS. 16A-16B provide a non-limiting illustration of such a patch. The patch may be configured to transmit, wirelessly, data collected by the patch to one or more external devices for further processing. In other embodiments, the single ultrasound probe may be embodied in a pill that may be swallowed by a patient. The pill may be configured to transmit, wirelessly, data collected by the ultrasound probe within the pill to one or more external devices for further processing. FIG. 17 illustrates a non-limiting example of such a pill.

The TX circuitry 904 (if included) may, for example, generate pulses that drive the individual elements of, or one or more groups of elements within, the transducer array(s) 902 so as to generate acoustic signals to be used for imaging. The RX circuitry 906, on the other hand, may receive and process electronic signals generated by the individual elements of the transducer array(s) 902 when acoustic signals impinge upon such elements.

In some embodiments, the timing & control circuit 908 may be, for example, responsible for generating all timing and control signals that are used to synchronize and coordinate the operation of the other elements in the device 900. In the example shown, the timing & control circuit 908 is driven by a single clock signal CLK supplied to an input port 916. The clock signal CLK may be, for example, a high-frequency clock used to drive one or more of the on-chip circuit components. In some embodiments, the clock signal CLK may, for example, be a 1.5625 GHz or 2.5 GHz clock used to drive a high-speed serial output device (not shown in FIG. 9) in the signal conditioning/processing circuit 110, or a 20 Mhz, 40 MHz, 100 MHz, 200 MHz, 250 MHz, 500 MHz, 750 MHz, or 1000 MHz clock used to drive other digital components on the die 912, and the timing & control circuit 1908 may divide or multiply the clock CLK, as necessary, to drive other components on the die 912. In other embodiments, two or more clocks of different frequencies (such as those referenced above) may be separately supplied to the timing & control circuit 908 from an off-chip source.

The power management circuit 918 may be, for example, responsible for converting one or more input voltages V_{IN} from an off-chip source into voltages needed to carry out operation of the chip, and for otherwise managing power consumption within the device 900. In some embodiments, for example, a single voltage (e.g., 0.4V, 0.9V, 1.5V, 1.8V, 2.5V, 3.3V, 5V, 12V, 80V, 100V, 120V, etc.) may be supplied to the chip and the power management circuit 918 may step that voltage up or down, as necessary, using a charge pump circuit or via some other DC-to-DC voltage conversion mechanism. In other embodiments, multiple different voltages may be supplied separately to the power management circuit 918 for processing and/or distribution to the other on-chip components.

As shown in FIG. 9, in some embodiments, a high intensity focused ultrasound (HIFU) controller 920 may be integrated on the die 912 so as to enable the generation of HIFU signals via one or more elements of the transducer array(s) 902. In other embodiments, a HIFU controller for driving the transducer array(s) 902 may be located off-chip, or even within a device separate from the device 900. That is, aspects of the present disclosure relate to provision of ultrasound-on-a-chip HIFU systems, with and without ultrasound imaging capability. It should be appreciated, however, that some embodiments may not have any HIFU capabilities and thus may not include a HIFU controller 920.

Moreover, it should be appreciated that the HIFU controller 920 may not represent distinct circuitry in those embodiments providing HIFU functionality. For example, in some embodiments, the remaining circuitry of FIG. 9 (other than the HIFU controller 920) may be suitable to provide ultrasound imaging functionality and/or HIFU, i.e., in some embodiments the same shared circuitry may be operated as an imaging system and/or for HIFU. Whether or not imaging or HIFU functionality is exhibited may depend on the power provided to the system. HIFU typically operates at higher powers than ultrasound imaging. Thus, providing the system a first power level (or voltage level) appropriate for imaging applications may cause the system to operate as an imaging system, whereas providing a higher power level (or voltage level) may cause the system to operate for HIFU. Such power management may be provided by off-chip control circuitry in some embodiments.

In addition to using different power levels, imaging and HIFU applications may utilize different waveforms. Thus, waveform generation circuitry may be used to provide suitable waveforms for operating the system as either an imaging system or a HIFU system.

In some embodiments, the system may operate as both an imaging system and a HIFU system (e.g., capable of providing image-guided HIFU). In some such embodiments, the same on-chip circuitry may be utilized to provide both functions, with suitable timing sequences used to control the operation between the two modalities.

In the example shown, one or more output ports 914 may output a high-speed serial data stream generated by one or more components of the signal conditioning/processing circuit 910. Such data streams may be, for example, generated by one or more USB 2.0, 3.0 and 3.1 modules, and/or one or more 1 Gb/s, 10 Gb/s, 40 Gb/s, or 100 Gb/s Ethernet modules, integrated on the die 912. In some embodiments, the signal stream produced on output port 914 can be fed to a computer, tablet, or smartphone for the generation and/or display of 2-dimensional, 3-dimensional, and/or tomographic images. It should be appreciated that the listed images are only examples of possible image types. Other examples may include 1-dimensional images, 0-dimensional spectral Doppler images, and time-varying images, including images combing 3D with time (time varying 3D images). In embodiments in which image formation capabilities are incorporated in the signal conditioning/processing circuit 910, even relatively low-power devices, such as smartphones or tablets which have only a limited amount of processing power and memory available for application execution, can display images using only a serial data stream from the output port 914. As noted above, the use of on-chip analog-to-digital conversion and a high-speed serial data link to offload a digital data stream is one of the features that helps facilitate an “ultrasound on a chip” solution according to some embodiments of the technology described herein.

Device 900 such as that shown in FIG. 9 may be used in any of a number of imaging and/or treatment (e.g., HIFU) applications, and the particular examples discussed herein should not be viewed as limiting. In one illustrative implementation, for example, an imaging device including an N×M planar or substantially planar array of PMUT elements may itself be used to acquire an ultrasonic image of a subject, e.g., a person's abdomen, by energizing some or all of the elements in the array(s) 902 (either together or individually) during one or more transmit phases, and receiving and processing signals generated by some or all of the elements in the array(s) 902 during one or more receive phases, such that during each receive phase the PMUT elements sense acoustic signals reflected by the subject. In other implementations, some of the elements in the array(s) 902 may be used only to transmit acoustic signals and other elements in the same array(s) 902 may be simultaneously used only to receive acoustic signals. Moreover, in some implementations, a single imaging device may include a P×Q array of individual devices, or a P×Q array of individual N×M planar arrays of PMUT elements, which components can be operated in parallel, sequentially, or according to some other timing scheme so as to allow data to be accumulated from a larger number of PMUT elements than can be embodied in a single device 900 or on a single die 912.

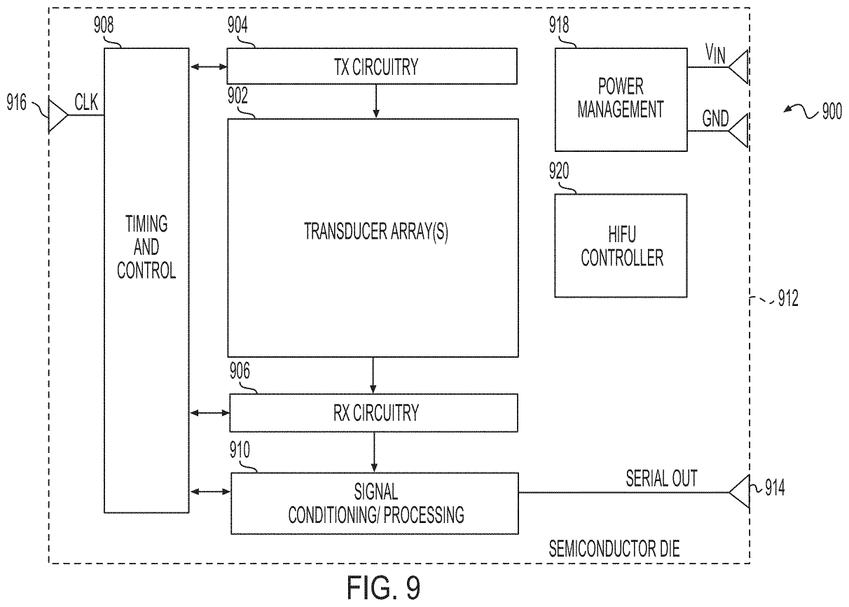

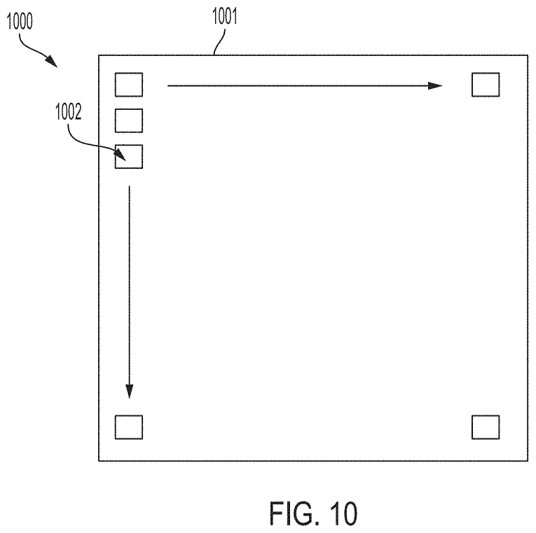

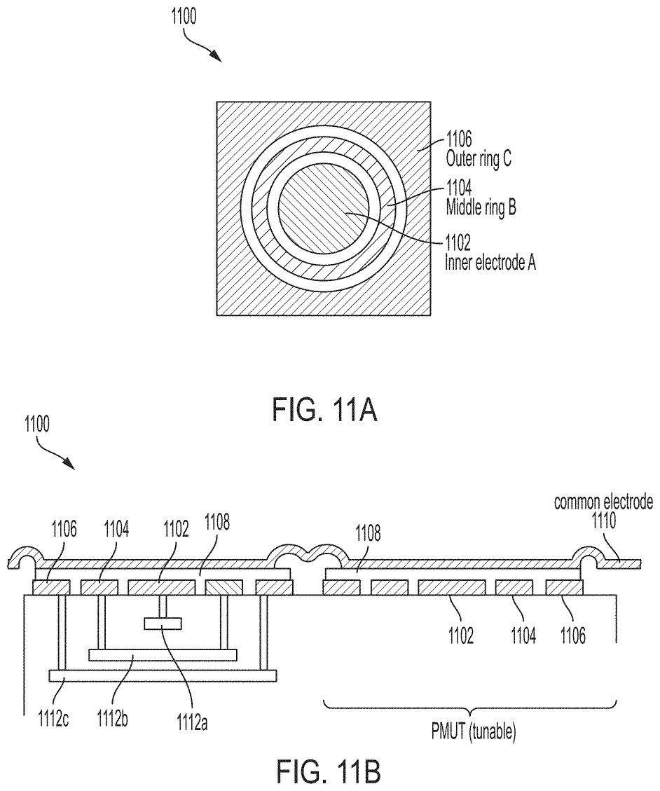

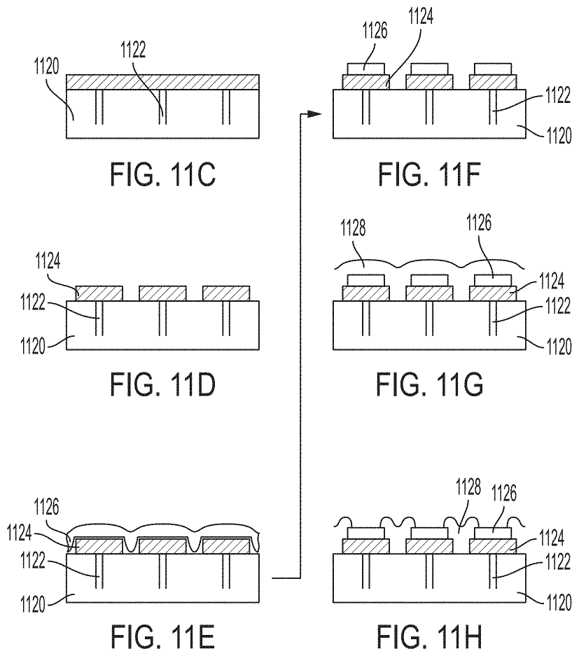

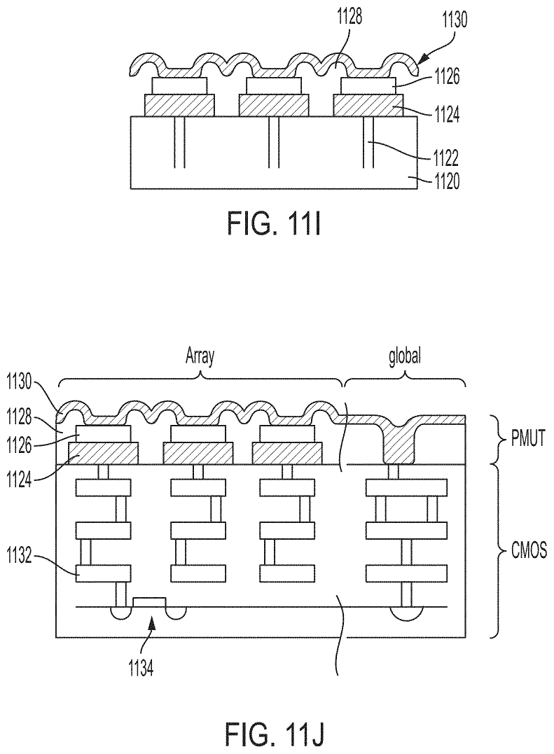

In some embodiments, an ultrasound device may include a transducer array, such as the transducer array 902 shown in FIG. 9, having an array of PMUTs where individual PMUTs are capable of emitting multiple frequencies. FIG. 10 illustrates a planar view of transducer array 1000 having ultrasonic transducers 1002, which are capable of emitting multiple frequencies. Thus, a multi-frequency PMUT array may be provided in some embodiments. The ultrasonic transducers 1002 may be disposed on a common substrate 1001. For example, the ultrasonic transducers 1002 may be PMUTs monolithically integrated with a semiconductor substrate. In some embodiments, the ultrasonic transducers 1002 may be PMUTs, and each may be configured to emit multiple frequencies. In other embodiments, one or more of the ultrasonic transducers 1002 may be configured to emit multiple frequencies. FIGS. 11A and 11B illustrate a non-limiting example of a PMUT configured to emit multiple frequencies.

FIG. 11A is a plan view of a PMUT 1100 having multiple electrodes 1102 (inner electrode A), 1104 (middle ring B), and 1106 (outer ring C), configured to provide selectable frequency operation of the PMUT. The illustrated electrodes may represent bottom electrodes of the PMUT. FIG. 11B is a cross-sectional view of the PMUT shown in FIG. 11A, where electrodes 1102, 1104, 1106 are formed under piezoelectric layer 1108, which is formed under a common electrode 1110. PMUT 1100 may be implemented as a transducer 1002 in transducer array 1000. The electrodes 1102, 1104, 1106 may be suitably sized and shaped to allow for PMUT 1100 to emit different discrete frequencies, or different frequency bands centered on respective peak frequencies. The area of the piezoelectric material that a particular electrode surrounds dictates the frequency emitted by the PMUT when the electrode is operated. To achieve high frequency, just inner electrode A may be driven, for example by signal line 1112a. To achieve middle (or intermediate) frequencies, inner electrode A and middle ring B may be driven, for example by signal lines 1112a and 1112b. To achieve low frequencies, the inner electrode A, middle ring B, and outer ring C may be driven, for example by signal lines 1112a, 1112b, and 1112c. The electrodes not being driven may be held at a constant bias. Electrode shapes other than those shown may be used.

It should be appreciated that FIGS. 11A and 11B illustrate a configuration in which the bottom electrodes are patterned and isolated, while the top electrode is a common electrode. This configuration may maximize fill factor and simplify making connection to the individual PMUTs. Such a configuration may be achieved using various processing techniques. As a non-limiting example, the following process flow may be used to achieve a configuration like that shown in FIGS. 11A-11B: deposit metallurgy on a substrate comprising circuitry and pattern the deposited metallurgy into signal islands (which are connected or coupled to the circuitry); deposit and pattern one or more piezoelectric films; and deposit common metallurgy over the structure which connects back to a common node. However, in other embodiments the top electrodes may be patterned and the bottom electrode may be a common electrode.

FIGS. 11C-11H illustrate a fabrication sequence for fabricating PMUTs according to a non-limiting embodiment. Such PMUTs may be used in an ultrasonic transducer array. FIGS. 11I-11J illustrate PMUT arrays according to a non-limiting embodiment.

FIG. 11C shows a starting point for the fabrication of a PMUT according to a non-limiting embodiment. A substrate 1120 has vias 1122 formed therein, and a metal layer 1124 is disposed on top of the substrate 1120. The substrate 1120 may be a CMOS substrate in some embodiments, and may include additional layering and circuitry, not shown for simplicity of illustration. For example, the substrate 1120 may include transistors and signal lines of the types shown in FIG. 11B. The vias 1122 may be formed of a conductive material, such as metal or conductive semiconductor. The metal layer 1124 may serve as an electrode for a PMUT, and may be any suitable material and thickness for this purpose. In some embodiments, the metal layer 1124 may be made of a conductive material other than metal.

As shown in FIG. 11D, the metal layer 1124 may be patterned to form discrete electrodes. The number, size, shape, and positioning of the electrodes may be chosen to provide desired operation. For example, the metal layer 1124 may be patterned into an array of tens, hundreds, or thousands of electrodes. Also, as described above in connection with FIGS. 11A-11B, multiple electrodes may be provided for a given PMUT, or in other embodiments a single electrode may be provided for a PMUT.

As shown in FIG. 11E, a piezoelectric layer 1126 may be formed over the top surface of the substrate 1120, including over the metal layer 1124. The piezoelectric material may be deposited conformally using any suitable technique for conformal deposition, including any of the techniques described herein relating to formation of piezoelectric thin films. The material and thickness of the piezoelectric layer 1126 may be selected to provide desired piezoelectric transducing behavior.

In FIG. 11F, the piezoelectric layer 1126 may be patterned to form discrete transducing regions aligned with the previously patterned electrodes. In the illustrated example, the patterned piezoelectric regions are narrower than underlying electrodes. However, this feature may not be included in all embodiments, as alternatives are possible.

In FIG. 11G, a dielectric layer 1128 may be deposited over the existing structure. The dielectric may be conformally deposited using any suitable technique for conformal deposition. The dielectric may be silicon oxide or any other suitable dielectric material. The dielectric layer 1128 may have any suitable thickness.

In FIG. 11H, the dielectric layer 1128 may be patterned as shown using any suitable lithography and etching techniques. In FIG. 11I, a metal layer 1130 may be deposited. The metal layer 1130 may be configured as a common electrode for the PMUTs. In alternative embodiments, the metal layer 1130 may be formed of a conductive material other than metal.

FIG. 11J illustrates an array of PMUTs monolithically integrated on an integrated circuit substrate 1102, which may be a CMOS substrate as explained above. Thus, FIG. 11J may represent an expanded view of the structure of FIG. 11I, showing additional signal lines 1132 and transistors 1134. As shown, the metal layer 1130 may be configured as a common electrode and may be connected to a global supply line. The array of PMUTs may be locally connected to suitable control circuitry, such as circuitry comprising the transistors 1134.

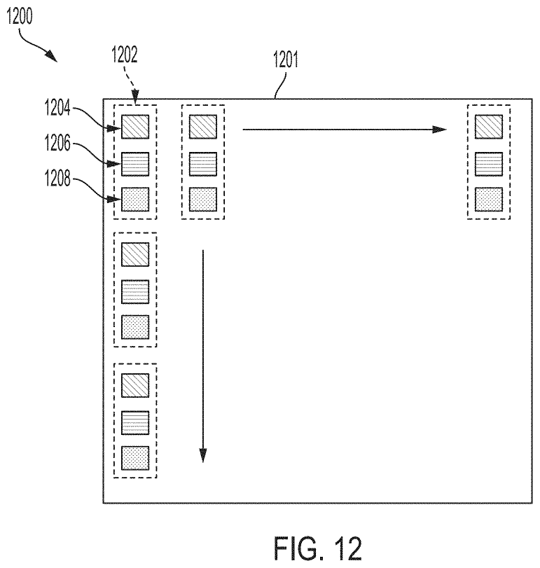

In some embodiments, an ultrasound device may include a transducer array, such as the transducer array 902 shown in FIG. 9, having an array of PMUTs arranged in sub-groups of PMUTs where individual PMUTs in a sub-group emit a different frequency than the other PMUTs in the sub-group. FIG. 12 illustrates a planar view of transducer array 1200 having PMUT sub-groups 1202 that include multiple PMUTs each capable of emitting a frequency different than the other PMUTs in the sub-group. As shown in FIG. 12, transducer array 1200 includes PMUTs arranged in sub-groups 1202, where a sub-group includes multiple PMUTs 1204, 1206, 1208 each configured to emit a different frequency, or different frequency band with respective peak frequencies. The different PMUTs and/or sub-groups of PMUTs may be arranged in any suitable manner in the transducer array, as the arrangement shown in FIG. 12 is a non-limiting example of a transducer array having sub-groups. The PMUTs may be formed on a substrate 1201. In some embodiments, the substrate 1201 is an integrated circuitry substrate (e.g., a CMOS substrate), and the PMUTs are monolithically integrated with the integrated circuit substrate.

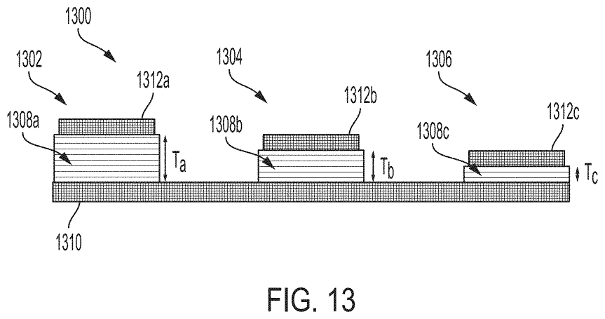

PMUTs in a sub-group, such as sub-groups 1202 shown in FIG. 12, may vary in a dimension and/or an amount of a piezoelectric material, which may account for the different frequencies the sub-group is configured to emit. In some embodiments, individual PMUTs in a sub-group have varying thicknesses. FIG. 13 is a cross-sectional view of a group of PMUTs 1300 having different thicknesses and frequencies of operation. The PMUTs in group 1300 include PMUT 1302, PMUT 1304, and PMUT 1306, each having different thicknesses of piezoelectric material 1308a, 1308b, 1308c, respectively. PMUT 1302 has a thickness Ta, PMUT 1304 has a thickness Tb less than Ta, and PMUT 1306 has a thickness Tc less than Tb. The PMUTs shown in sub-group 1300 share bottom electrode 1310 and have separate top electrodes 1312a, 1312b, 1312c. The frequency that a PMUT emits may depend on the thickness of the piezoelectric material, such that PMUTs having smaller thicknesses of piezoelectric material emit higher frequencies than PMUTs having greater thicknesses of piezoelectric material. For example, PMUT 1302 has piezoelectric material 1308a that has a greater thickness than piezoelectric material 1308c of PMUT 1306. In this example, PMUT 1306 is configured to emit a higher frequency during operation than PMUT 1302.

The provision of a frequency tunable or selectable PMUT array may provide for a “universal” ultrasound probe, capable of operating across a frequency range conventionally implicating multiple different ultrasound probes. That is, the ultrasound devices described herein may operate across a greater frequency range than conventional devices, thus allowing for shallow and deep imaging.

Forms of Universal Ultrasound Device

Ultrasound devices of the types described herein may be embodied in various form factors. For example, ultrasound probes, stethoscopes, patches, pills, or other form factors may include or implement one or more of the aspects described herein. Various non-limiting examples are now described.

A universal ultrasound device may be implemented in any of a variety of physical configurations including, for example, as a part of an internal imaging device, such as a pill to be swallowed by a subject or a pill mounted on an end of a scope or catheter, as part of a handheld device including a screen to display obtained images, as part of a patch configured to be affixed to the subject, or as part of a hand-held probe.

In some embodiments, a universal ultrasound probe may be embodied in a handheld device 1402 illustrated in FIGS. 14A and 14B. Handheld device 1402 may be held against (or near) a subject 1400 and used to image the subject. Handheld device 1402 may comprise an ultrasound probe (e.g., a universal ultrasound probe) and display 1404, which in some embodiments, may be a touchscreen. Display 1404 may be configured to display images of the subject generated within handheld device 1402 using ultrasound data gathered by the ultrasound probe within device 1402.

In some embodiments, handheld device 1402 may be used in a manner analogous to a stethoscope. A medical professional may place handheld device 1402 at various positions along a patient's body. The ultrasound probe within handheld device 1402 may image the patient. The data obtained by the ultrasound probe may be processed and used to generate image(s) of the patient, which image(s) may be displayed to the medical professional via display 1404. As such, a medical professional could carry hand-held device (e.g., around their neck or in their pocket) rather than carrying around multiple conventional probes, which is burdensome and impractical.

In some embodiments, a universal ultrasound probe may be embodied in hand-held probe 1500 shown in FIG. 15. Hand-held probe 1500 may be configured to transmit data collected by the probe 1500 wirelessly to one or more external host devices (not shown in FIG. 15) for further processing. In other embodiments, hand-held probe 1500 may be configured transmit data collected by the probe 1500 to one or more external devices using one or more wired connections, as aspects of the technology described herein are not limited in this respect.

In some embodiments, a universal ultrasound probe may be embodied in a patch that may be coupled to a patient. For example, FIGS. 16A and 16B illustrate a patch 1610 coupled to patient 1612. The patch 1610 may be configured to transmit, wirelessly for example, data collected by the patch 1610 to one or more external devices (not shown) for further processing. For purposes of illustration, a top housing of the patch 1610 is depicted in a transparent manner to depict exemplary locations of various internal components of the patch.

Patch 1610 may include a circuit board configured to support various components, such as for example a heat sink, battery, and communications circuitry. In one embodiment, communication circuitry of the patch 1610 includes one or more short- or long-range communication platforms. Exemplary short-range communication platforms include Bluetooth (BT), Bluetooth Low Energy (BLE), Near-Field Communication (NFC). Long-range communication platforms include Wi-Fi and Cellular. While not shown, the communication platform may include a front-end radio, antenna and other processing circuitry configured to communicate radio signal to an auxiliary device (not shown). The radio signal may include ultrasound imaging information obtained by patch 1610.

In an exemplary embodiment, communication circuitry transmits periodic beacon signals according to IEEE 802.11 and other prevailing standards. The beacon signal may include a BLE advertisement. Upon receipt the beacon signal or the BLE advertisement, an auxiliary device (not shown) may respond to patch 1610. That is, the response to the beacon signal may initiate a communication handshake between patch 1610 and the auxiliary device.

The auxiliary device may include a laptop, desktop, smartphone or any other device configured for wireless communication. The auxiliary device may act as a gateway to cloud or Internet communication. In an exemplary embodiment, the auxiliary device may include the patient's own smart device (e.g., smartphone) which communicatively couples to patch 1610 and periodically receives ultrasound information from patch 1610. The auxiliary device may then communicate the received ultrasound information to external sources.

A circuit board of the patch 1610 may comprise one or more processing circuits, including one or more controllers to direct communication through the communication circuitry. For example, the circuit board may engage communication circuitry periodically or on as-needed basis to communicate information with one or more auxiliary devices. Ultrasound information may include signals and information defining an ultrasound image captured by patch 1610. Ultrasound information may also include control parameters communicated from the auxiliary device to patch 1610. The control parameters may dictate the scope of the ultrasound image to be obtained by patch 1610.

In one embodiment, the auxiliary device may store ultrasound information received from patch 1610. In another embodiment, the auxiliary device may relay ultrasound information received from patch 1610 to another station. For example, the auxiliary device may use Wi-Fi to communicate the ultrasound information received from patch 1610 to a cloud-based server. The cloud-based server may be a hospital server or a server accessible to the physician directing ultrasound imaging. In another exemplary embodiment, patch 1610 may send sufficient ultrasound information to the auxiliary device such that the auxiliary device may construct an ultrasound image therefrom. In this manner, communication bandwidth and power consumption may be minimized at patch 1610.

In still another embodiment, the auxiliary device may engage patch 1610 through radio communication (i.e., through communication circuitry) to actively direct operation of patch 1610. For example, the auxiliary device may direct patch 1610 to produce ultrasound images of the patient at periodic intervals. The auxiliary device may direct the depth of the ultrasound images taken by patch 1610. In still another example, the auxiliary device may control the manner of operation of the patch so as to preserve power consumption at a battery. Upon receipt of ultrasound information from patch 1610, the auxiliary device may operate to cease imaging, increase imaging rate or communicate an alarm to the patient or to a third party (e.g., physician or emergency personnel).

It should be noted that the communication platform described in relation with FIGS. 16A and 16B may also be implemented in other form-factors disclosed herein. For example, the communication platform (including control circuitry and any interface) may be implemented in the ultrasound pill as illustrated in FIG. 17, the handheld device as illustrated in FIGS. 14A-14B or the handheld probe as illustrated in FIG. 15.

In some embodiments, a universal ultrasound probe may be embodied in a pill to be swallowed by a subject. As the pill travels through the subject, the ultrasound probe within the pill may image the subject and wirelessly transmit obtained data to one or more external devices for processing the data received from the pill and generating one or more images of the subject. For example, as shown in FIG. 17, pill 1702 comprising an ultrasound probe may be configured to communicate wirelessly (e.g., via wireless link 1701) with external device 1700, which may be a desktop, a laptop, a handheld computing device, and/or any other device external to pill 1702 and configured to process data received from pill 1702. A person may swallow pill 1702 and, as pill 1702 travels through the person's digestive system, pill 1702 may image the person from within and transmit data obtained by the ultrasound probe within the pill to external device 1700 for further processing. In some embodiments, the pill 1702 may comprise an onboard memory and the pill 1702 may store the data on the onboard memory such that the data may be recovered from the pill 1702 once it has exited the person.

In some embodiments, a pill comprising an ultrasound probe may be implemented by potting the ultrasound probe within an outer case. In some embodiments, a pill comprising an ultrasound probe may be implemented by encasing the ultrasound probe within an outer housing. In some embodiments, the ultrasound probe implemented as part of a pill may comprise one or multiple ultrasonic transducer (e.g., PMUT) arrays, one or more image reconstruction chips, an FPGA, communications circuitry, and one or more batteries.

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. The transitional phrases “consisting of” and “consisting essentially of” shall be closed or semi-closed transitional phrases, respectively.

What is claimed is:

1. An ultrasound device, comprising:
an integrated multi-substrate die including a piezoelectric micromachined ultrasonic transducer (PMUT) substrate, a transmit circuitry substrate, and a receive circuitry substrate coupled together.

2. The ultrasound device of claim 1, wherein the transmit circuitry substrate comprises transmit circuitry and the receive circuitry substrate comprises receive circuitry implemented in a different node than the transmit circuitry.

3. The ultrasound device of claim 2, wherein the transmit circuitry is implemented in a larger node than the receive circuitry.

4. The ultrasound device of claim 3, wherein the transmit circuitry is configured to implement voltages greater than those implemented by the receive circuitry.

5. The ultrasound device of claim 1, wherein the PMUT substrate comprises a frequency tunable PMUT array.

6. The ultrasound device of claim 5, wherein the frequency tunable PMUT array comprises PMUTs of different dimensions.

7. The ultrasound device of claim 6, wherein the PMUTs of different dimensions have different thicknesses.

8. The ultrasound device of claim 6, wherein the frequency tunable PMUT array comprises a PMUT having multiple excitation electrodes of different dimensions.

9. The ultrasound device of claim 8, wherein the PMUT having multiple excitation electrodes of different dimensions has a first electrode configured to excite a first area of the PMUT and a second electrode configured to excite a second area of the PMUT greater than the first area.

10. The ultrasound device of claim 9, wherein the transmit circuitry substrate comprises transmit circuitry configured to individually excite the first electrode or the second electrode.

11. An ultrasound device, comprising:
an integrated multi-substrate die including a piezoelectric micromachined ultrasonic transducer (PMUT) substrate coupled with an integrated circuit (IC) substrate.

12. The ultrasound device of claim 11, wherein the IC substrate is a complementary metal oxide semiconductor (CMOS) substrate comprising CMOS circuitry.

13. The ultrasound device of claim 11, wherein the PMUT substrate comprises a frequency tunable PMUT array.

14. The ultrasound device of claim 13, wherein the frequency tunable PMUT array comprises PMUTs of different dimensions.

15. The ultrasound device of claim 14, wherein the PMUTs of different dimensions have different thicknesses.

16. The ultrasound device of claim 13, wherein the frequency tunable PMUT array comprises a PMUT having multiple excitation electrodes of different dimensions.

17. The ultrasound device of claim 16, wherein the PMUT having multiple excitation electrodes of different dimensions has a first electrode configured to excite a first area of the PMUT and a second electrode configured to excite a second area of the PMUT greater than the first area.

18. The ultrasound device of claim 17, wherein the IC substrate comprises transmit circuitry configured to individually excite the first electrode or the second electrode.

19. An ultrasound device, comprising:
a substrate
an integrated circuit formed in the substrate; and
a layer of thin film piezoelectric micromachined ultrasonic transducers (PMUTs) integrated with the substrate.

20. The ultrasound device of claim 19, wherein the thin film PMUTs of the layer of thin film PMUTs lack a transducing gap.

21. The ultrasound device of claim 19, wherein the thin film PMUTs of the layer of thin film PMUTs are frequency tunable.

22. The ultrasound device of claim 21, wherein a thin film PMUT of the layer of thin film PMUTs includes multiple selectable electrodes configured to excite different regions of the PMUT.
